(19) United States
(12) Patent Application Publication
(10) Pub. No.: US 2025/0057991 A1
(43) Pub. Date: Feb. 20, 2025

(54) NANOMATERIAL COMPOSITION AND USE OF THE SAME FOR MAGNETIC FIELD-INDUCED ELECTRICAL STIMULATION OF CELLS

(71) Applicant: National Yang Ming Chiao Tung University, Hsinchu City (TW)

(72) Inventors: Po-Han Chiang, Hsinchu City (TW); Chao-Chun Cheng, Hsinchu City (TW)

(21) Appl. No.: 18/540,750

(22) Filed: Dec. 14, 2023

(30) Foreign Application Priority Data
Aug. 14, 2023 (TW) 112130503

(51) Int. Cl. A61K 49/18; A61N 1/36

(52) U.S. Cl. CPC A61K 49/183; A61N 1/36

(57) ABSTRACT

A nanomaterial composition for magnetic field-induced electrical stimulation of cells includes a piezoelectric nanoparticle and a magnetic nanodisc. The piezoelectric nanoparticle is conjugated to a first molecule of a specific binding molecule pair and is coated with a cell-binding molecule. The magnetic nanodisc is conjugated to a second molecule of the specific binding molecule pair and is attached to the piezoelectric nanoparticle through bonding of the second molecule and the first molecule. The magnetic nanodisc converts a magnetic energy into a mechanical energy in the presence of an external magnetic field, and the mechanical energy is then applied to the piezoelectric nanoparticle that is in contact with the cells via the cell-binding molecule, such that the piezoelectric nanoparticle converts the mechanical energy into an electrical energy, so as to electrically stimulate the cells. A method for magnetic field-induced electrical stimulation of cells in a subject is also provided.

### CROSS-REFERENCE TO RELATED APPLICATION

This application claims priority to Taiwanese Invention Patent Application No. 112130503, filed on Aug. 14, 2023, and incorporated by reference herein in its entirety.

### FIELD

The present disclosure relates to a nanomaterial composition for magnetic field-induced electrical stimulation of cells. The present disclosure also relates to use of the nanomaterial composition for magnetic field-induced electrical stimulation of cells in a subject.

### BACKGROUND

Electrical stimulation is not only used for treating various nerve and muscle-related disorders or conditions (for example, spinal cord injury, muscular dystrophy, amyotropic lateral sclerosis, multiple system atrophy, chronic pain, schizophrenia, Parkinson's disease, Alzheimer's disease, Huntington's disease, epilepsy, depression and bipolar disorder), but also is widely applied in other types of disorders, such as gastrointestinal disorders, as reported in Payne S. C. et al. (2019), Nat. Rev. Gastroenterol. Hepatol., 16: 89-105, and cancers, as reported in Das R. et al. (2021), Front. Bioeng. Biotechnol., 9: 795300.

Electrodes used clinically for performing electrical stimulation are usually wired and have a large size, causing inconvenience during use thereof. In addition, there is a risk of infection when such electrodes are surgically implanted into the body, and displacement of the electrodes might occur after implantation due to daily activities. Therefore, those skilled in the art endeavor to develop electrical stimulation methods which do not require surgery and which can be conducted in a wireless manner.

Kozielski K. L. et al. (2021), Sci. Adv., 7: eabc4189 discloses injectable nanoelectrodes for wireless deep brain stimulation which are particles each having a core-shell structure and which are formed by coating magnetostrictive CoFe₂O₄ nanoparticles with piezoelectric material, i.e., BaTiO₃. The injectable nanoelectrodes have been proven using in vitro and in vivo experiments to be capable of outputting electrical signals and modulating neuronal activity via magnetoelectric effect in response to application of a magnetic field.

In spite of the aforesaid, the preparation of such injectable nanoelectrodes, which requires the piezoelectric material to be coated on the magnetostrictive CoFe₂O₄ nanoparticles, is relatively difficult, and since magnetic force generated by magnetostriction is relatively small, a magnetic field with relatively high frequency and relatively high amplitude are usually required during use of the injectable nanoelectrodes.

### SUMMARY

Therefore, an object of the present disclosure is to provide a nanomaterial composition for magnetic field-induced electrical stimulation of cells, and a method for performing magnetic field-induced electrical stimulation of cells in a subject which can alleviate at least one of the drawbacks of the prior art.

According to one aspect of the present disclosure, the nanomaterial composition for magnetic field-induced electrical stimulation of cells includes a piezoelectric nanoparticle and a magnetic nanodisc. The piezoelectric nanoparticle is conjugated to a first molecule of a specific binding molecule pair and is coated with a cell-binding molecule. The magnetic nanodisc is conjugated to a second molecule of the specific binding molecule pair and is attached to the piezoelectric nanoparticle through bonding of the second molecule and the first molecule. The magnetic nanodisc converts a magnetic energy into a mechanical energy in the presence of an external magnetic field, and the mechanical energy is then applied to the piezoelectric nanoparticle that is in contact with the cells via the cell-binding molecule, such that the piezoelectric nanoparticle converts the mechanical energy into an electrical energy, so as to electrically stimulate the cells.

According to another aspect of the present disclosure, the method for magnetic field-induced electrical stimulation of cells in a subject includes:
administering to a subject, the aforesaid nanomaterial composition, such that a piezoelectric nanoparticle, which is conjugated to a first molecule of a specific binding molecule pair and which is coated with a cell-binding molecule, is in contact with cells in the subject via the cell-binding molecule, and such that a magnetic nanodisc, which is conjugated to a second molecule of the specific binding molecule pair, is attached to the piezoelectric nanoparticle through bonding of the second molecule and the first molecule; and
applying an external magnetic field to the subject, such that the magnetic disc converts a magnetic energy into a mechanical energy, and the mechanical energy is then applied to the piezoelectric nanoparticle that is in contact with the cells, such that the piezoelectric nanoparticle converts the mechanical energy into an electrical energy, so as to electrically stimulate the cells.

### BRIEF DESCRIPTION OF THE DRAWINGS

Other features and advantages of the present disclosure will become apparent in the following detailed description of the embodiment(s) with reference to the accompanying drawings. It is noted that various features may not be drawn to scale.

FIG. 1 is a scanning electron microscope (SEM) image showing neutravidin-conjugated BaTiO₃ piezoelectric nanoparticle (n-BTO) and biotin-conjugated Fe₃O₄ magnetic nanodisc (b-MND) on primary hippocampal neurons of Example 1, infra.

FIG. 2 is a fluorescence microscope image showing n-BTO and b-MND on the primary hippocampal neurons of Example 1, infra.

FIG. 3 is graphs showing changes in fluorescence intensity measured over different times in each group of Example 2, infra, in which a magnetic field is applied for a time period ranging from the 50^{th} second to the 100^{th} second of the measurement.

FIG. 4A are immunofluorescence staining images of an amygdala on one side of the mouse brain which was administered with n-BTO and b-MND, and those of another amygdala on another side of the mouse brain which was not administered with n-BTO and b-MND of Example 3, infra.

FIG. 4B is a graph of the density of c-Fos-positive cells in the amygdala on one side of the mouse brain which was administered with n-BTO and b-MND in an amount ratio of 10:1 or 5:1 and that in the another amygdala on the another side of the mouse brain which was not administered with n-BTO and b-MND of Example 3, infra.

FIG. 5 is a graph showing the highest fluorescence intensity determined in each group after magnetic field-induced electrical stimulation in cells treated with n-BTO having different particle sizes in combination with b-MND₂₅₀ of Example 4, infra.

FIG. 6 is a graph showing the highest fluorescence intensity determined in each group after magnetic field-induced electrical stimulation in cells treated with n-BTO having different particle sizes in combination with b-MND having different diameters of Example 4, infra.

### DETAILED DESCRIPTION

Before the present disclosure is described in greater detail, it should be noted that if any prior art publication is referred to herein, such reference does not constitute an admission that the publication forms a part of the common general knowledge in the art, in Taiwan or any other country.

For the purpose of this specification, it will be clearly understood that the word “comprising” means “including but not limited to”, and that the word “comprises” has a corresponding meaning.

Unless otherwise defined, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

In order to address the current limitations of wireless electrical stimulation methods in cells, the applicants surprisingly found that a combination of two different types of nanomaterials, i.e., piezoelectric nanoparticle and magnetic nanodisc each in a specified dimension, when administered to cells under a weak magnetic field, is capable of electrically stimulate the cells so as to activate the same.

Therefore, the present disclosure provides a nanomaterial composition for magnetic field-induced electrical stimulation of cells which includes a piezoelectric nanoparticle and a magnetic nanodisc. The piezoelectric nanoparticle is conjugated to a first molecule of a specific binding molecule pair and is coated with a cell-binding molecule. The magnetic nanodisc is conjugated to a second molecule of the specific binding molecule pair and is attached to the piezoelectric nanoparticle through bonding of the second molecule and the first molecule. The magnetic nanodisc converts a magnetic energy into a mechanical energy (i.e., generation of torque) in the presence of an external magnetic field, and the mechanical energy is then applied to the piezoelectric nanoparticle that is in contact with the cells via the cell-binding molecule, such that the piezoelectric nanoparticle converts the mechanical energy into an electrical energy, so as to electrically stimulate the cells.

As used herein, the term “magnetic field-induced electrical stimulation” can be used interchangeably with the term “magnetoelectric stimulation”, and is intended to refer to generation of an electrical energy under a magnetic field and introduction of the electrical energy into target cells.

According to the present disclosure, the target cells are not particularly limited, and may include cells in various tissue, organs or systems in different parts of the body where any conventional electrical stimulation techniques may be applied (for example, electrical stimulation of the brain, spinal cord, muscles, peripheral nerves, gastrointestinal tract, cancers or tumors, osteocytes, etc.) to regulate physiological activity or to treat diseases (for example, regulating neural activity; promoting nerve or muscle regeneration, such as repair of spinal cord injury, muscular dystrophy, amyotrophic lateral sclerosis, and multiple system atrophy; relieving pain, such as chronic pain; improving neurological diseases, such as schizophrenia, Parkinson's diseases, Alzheimer's disease, Huntington's disease, epilepsy, depression, and bipolar disorder; and improving gastrointestinal disorders, such as obesity, gastroesophageal reflux, and inflammatory bowel disease), and are expected to achieve the same results after electrical stimulation.

Examples of the cells, which can be electrically stimulated, may include, but are not limited to, neuronal cells, cancer cells, osteocytes, vascular endothelial cells, muscle cells, and peritoneal mesothelial cells. In an exemplary embodiment, the cells, which are electrically stimulated, are neuronal cells.

According to the present disclosure, the type of the piezoelectric nanoparticle is not particularly limited, and the piezoelectric nanoparticle may be obtained commercially, or may be prepared using techniques well-known and customary to those skilled in the art. In this regard, those skilled in the art may refer to journal articles, e.g., Jordan T. et al. (2020), ACS Appl. Nano Mater., 3: 2636-2646, Orudzhev F. et al. (2020), Sensors, 20:6736, Fan C. H. et al. (2023), ACS Nano., 17: 9140-9154, and Marino A. et al. (2015), ACS Nano., 9: 7678-7689.

Examples of the piezoelectric nanoparticle may include, but are not limited to, BaTiO₃ nanoparticle, MoS₂ nanoparticle, KNbO₃ nanoparticle, LiNbO₃ nanoparticle, BiFeO₃ nanoparticle, and Pb(Zr, Ti)O₃ nanoparticle. In an exemplary embodiment, the piezoelectric nanoparticle is BaTiO₃ nanoparticle.

In certain embodiments, the piezoelectric nanoparticle may have a particle size ranging from 50 nm to 500 nm. In certain embodiments, the piezoelectric nanoparticle may have a particle size ranging from 50 nm to 200 nm. In an exemplary embodiment, the piezoelectric nanoparticle has a particle size of 50 nm. In another exemplary embodiment, the piezoelectric nanoparticle has a particle size of 100 nm.

According to the present disclosure, the type of the magnetic nanodisc is not particularly limited, and the magnetic nanodisc may be obtained commercially, or may be prepared using techniques well-known and customary to those skilled in the art. In this regard, those skilled in the art may refer to journal articles, e.g., Su C. L. et al. (2022), Commun. Biol., 5: 1161, and Gregurec D. et al. (2020), ACS Nano., 14: 8036-8045.

Examples of the magnetic nanodisc may include, but are not limited to, Fe₃O₄ nanodisc, γ-Fe₂O₃ nanodisc, CoFe₂O₄ nanodisc, Co_{1.2}Fe_{1.8}O₄ nanodisc, and Ni(OH)₂ nanodisc. In an exemplary embodiment, the magnetic nanodisc is Fe₃O₄ nanodisc.

In certain embodiments, the magnetic nanodisc may have a diameter ranging from 150 nm to 250 nm. In an exemplary embodiment, the magnetic nanodisc has a diameter of 250 nm. In another exemplary embodiment, the magnetic nanodisc has a diameter of 200 nm.

In certain embodiments, a ratio of a particle size of the piezoelectric nanoparticle to a diameter of the magnetic nanodisc ranges from 1:2.5 to 1:5.

In certain embodiments, a weight ratio of the piezoelectric nanoparticle to the magnetic nanodisc ranges from 1:0.02 to 1:10. In certain embodiments, the weight ratio of the piezoelectric nanoparticle to the magnetic nanodisc ranges from 1:0.1 to 1:10. In certain embodiments, the weight ratio of the piezoelectric nanoparticle to the magnetic nanodisc ranges from 1:0.1 to 1:1. In an exemplary embodiment, the weight ratio of the piezoelectric nanoparticle to the magnetic nanodisc is 1:0.1. In another exemplary embodiment, the weight ratio of the piezoelectric nanoparticle to the magnetic nanodisc is 1:1.

As used herein, the term “specific binding molecule pair” refers to two molecules (i.e., a first molecule and a second molecule) that are capable of specifically binding to each other. Examples of the specific binding molecule pair may include, but are not limited to, neutravidin-biotin pair, avidin-biotin pair, antigen-antibody pair, ligand-receptor pair, DNA-DNA pair, DNA-binding protein-DNA pair, and click chemistry reactants. In an exemplary embodiment, the first molecule of the specific binding molecule pair is neutravidin, and the second molecule of the specific binding molecule pair is biotin.

As used herein, the term “cell-binding molecule” refers to a biocompatible molecule that is capable of binding and/or attaching to the cells or being absorbed on the cells.

According to the present disclosure, the piezoelectric nanoparticle may be coated with the cell-binding molecule to allow the piezoelectric nanoparticle to be in contact with the cells via the cell-binding molecule. Examples of the cell-binding molecule may include, but are not limited to, a cell-specific antibody, and a cell-affinitive molecule.

Examples of the cell-specific antibody may include, but are not limited to, a neuronal cell-specific antibody (e.g., anti-A2B5 antibody and anti-Thy1 antibody), and a cancer cell-specific antibody (e.g., anti-CD146 antibody, anti-IL-6 antibody, and anti-EGFR antibody).

Examples of the cell-affinitive molecule may include, but are not limited to, methoxy polyethylene glycol (mPEG)-silane, polyethylene glycol (PEG)-NH₂, PEG-COOH, poly (maleic anhydride-alt-1-octadecene (PMAO), PMAO-PEG, and polyvinylpyrrolidone (PVP).

According to the present disclosure, conjugation of the piezoelectric nanoparticle to the first molecule of the specific binding molecule pair and conjugation of the magnetic nanodisc to the second molecule of the specific binding molecule pair may be conducted using techniques well-known and customary to those skilled in the art. In certain embodiments, the conjugation of the piezoelectric nanoparticle to the first molecule of the specific binding molecule pair and conjugation of the magnetic nanodisc to the second molecule of the specific binding molecule pair may be conducted by functionalizing the piezoelectric nanoparticle and the magnetic nanodisc with a functionalization agent. Examples of the functionalization agent may include, but are not limited to, methoxy polyethylene glycol (mPEG), and poly (maleic anhydride-alt-1-octadecene (PMAO). In an exemplary embodiment, the functionalization agent is mPEG. In another exemplary embodiment, the functionalization agent is PMAO.

According to the present disclosure, in order to achieve an excellent conjugation, the procedures and conditions for conducting the conjugation may vary depending on several factors, such as the type of the specific binding molecule pair, the type of the functionalization agent, etc., and determination of such procedures and conditions is within the routine skill of those skilled in the art.

The present disclosure also provides a method for magnetic field-induced electrical stimulation of cells in a subject which includes: administering to a subject, the aforesaid nanomaterial composition, such that the piezoelectric nanoparticle, which is conjugated to the first molecule of the specific binding molecule pair and which is coated with the cell-binding molecule, is in contact with cells in the subject via the cell-binding molecule, and such that the magnetic nanodisc, which is conjugated to the second molecule of the specific binding molecule pair, is attached to the piezoelectric nanoparticle through bonding of the second molecule and the first molecule; and applying an external magnetic field to the subject, such that the magnetic disc converts the magnetic energy into a mechanical energy (i.e., generation of torque), and the mechanical energy is then applied to the piezoelectric nanoparticle that is in contact with the cells, such that the piezoelectric nanoparticle converts the mechanical energy into the electrical energy, so as to electrically stimulate the cells.

According to the present disclosure, the cells, the piezoelectric nanoparticle, the magnetic nanodisc, and the specific binding molecule pair have been described in the foregoing, and are not repeated herein for the sake of brevity.

According to the present disclosure, application of the external magnetic field may be conducted using techniques well-known and customary to those skilled in the art. In this regard, those skilled in the art may refer to journal articles, e.g., Su C. L. et al. (2022), Commun. Biol., 5: 1161, and Gregurec D. et al. (2020), ACS Nano., 14: 8036-8045. It should be noted that the procedures and conditions for applying the external magnetic field may depend on various factors, such as the type, size, dosage ratio, etc. of the piezoelectric nanoparticle and the magnetic nanodisc of the nanomaterial composition, such that the magnetic nanodisc converts the magnetic energy into the mechanical energy (i.e., generates torque) which is then applied to the piezoelectric nanoparticle, thereby achieving an excellent electrical stimulation. The determination of the procedures and conditions for applying the external magnetic field is within the routine skill of those skilled in the art.

According to the present disclosure, the external magnetic field may be an alternating magnetic field.

According to the present disclosure, the external magnetic field may have an amplitude of not greater than 200 mT. In certain embodiments, the external magnetic field may have an amplitude ranging from 1 mT to 200 mT. In certain embodiments, the external magnetic field may have an amplitude ranging from 25 mT to 100 mT. In an exemplary embodiment, the external magnetic field has an amplitude of 50 mT.

According to the present disclosure, the external magnetic field may have a frequency of not greater than 140 Hz. In certain embodiments, the external magnetic field may have a frequency ranging from 1 Hz to 140 Hz. In certain embodiments, the external magnetic field may have a frequency ranging from 5 Hz to 20 Hz. In an exemplary embodiment, the external magnetic field has a frequency of 10 Hz.

As used herein, the term “subject” refers to any animal of interest, such as humans, monkeys, cows, sheep, horses, pigs, goats, dogs, cats, mice, and rats. In certain embodiments, the subject is a human.

As used herein, the term “administering” can be used interchangeably with the term “administration”, and means introducing, providing or delivering a pre-determined active ingredient to a subject by any suitable routes to perform its intended function.

According to the present disclosure, the piezoelectric nanoparticle and the magnetic nanodisc of the nanomaterial composition may be combined to be co-administered to the subject. In certain embodiments, the piezoelectric nanoparticle and the magnetic nanodisc of the nanomaterial composition may be combined into a single dosage form to be administered to the subject. In certain embodiments, the piezoelectric nanoparticle and the magnetic nanodisc of the nanomaterial composition may be administered to the subject in separate dosage forms. In certain embodiments, the piezoelectric nanoparticle and the magnetic nanodisc of the nanomaterial composition may be alternately or sequentially administered to the subject with a predetermined time interval between administration of the piezoelectric nanoparticle and administration of the magnetic nanodisc. According to the present disclosure, the predetermined time interval between administration of the piezoelectric nanoparticle and administration of the magnetic nanodisc may be adjusted according to practical requirements.

In certain embodiments, the piezoelectric nanoparticle and the magnetic nanodisc of the nanomaterial composition are simultaneously administered to the subject.

In certain embodiments, the piezoelectric nanoparticle and the magnetic nanodisc of the nanomaterial composition are sequentially administered to the subject.

According to the present disclosure, the nanomaterial composition may be formulated as a pharmaceutical composition.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the pharmaceutically acceptable carrier are within the expertise of those skilled in the art.

According to the present disclosure, the pharmaceutical composition may be made into a dosage form suitable for parenteral administration (including an injection, e.g., a sterile aqueous solution, or a dispersion) using technology well-known to those skilled in the art, and may be administered via one of the following routes: intraperitoneal injection, intrapleural injection, intramuscular injection, intravenous injection, intraarterial injection, intraarticular injection, intrasynovial injection, intrathecal injection, intracranial injection, intraepidermal injection, subcutaneous injection, intradermal injection, intralesional injection, and sublingual administration.

The present disclosure will be described by way of the following examples. However, it should be understood that the following examples are intended solely for the purpose of illustration and should not be construed as limiting the present disclosure in practice.

### EXAMPLES

### General Experimental Materials

### 1. BaTiO₃ Piezoelectric Nanoparticle (Hereinafter “BTO”)

BTO having different particle sizes (i.e., 50 nm, 100 nm, 200 nm, 300 nm and 500 nm) used in the following experiments were purchased from US Research Nanomaterials, Inc.

2. Fe₃O₄ Magnetic Nanodisc (Hereinafter “MND”)

MND having different diameters (i.e., 150 nm, 200 nm and 250 nm) used in the following experiments were synthesized according to the methods described in Su C. L. et al. (2022), Commun. Biol., 5: 1161, and Gregurec D. et al. (2020), ACS Nano., 14: 8036-8045. In brief, 0.273 g of FeCl₃·6H₂O (Sigma-Aldrich), 10 ml of 99.5% ethanol, water in a volume ranging from 0.6 mL to 1.0 mL (1.0 mL, 0.8 mL and 0.6 ml of water were used for MND having diameters of 150 nm, 200 nm and 250 nm, respectively), and 0.8 g of anhydrous sodium acetate) were mixed and then heated at 180° C. for 18 hours so as to obtain non-magnetic hematite nanodisc. Thereafter, 1 mg of the non-magnetic hematite nanodisc was mixed with 20 ml of tri-octylamine (Acros Organics) and 1 g of oleic acid (Sigma-Aldrich), followed by a reduction reaction conducted by heating at 370° C. for 25 minutes in an atmosphere containing 5% of H₂ and 95% of N₂, thereby obtaining the MND.

### 3. Experimental Animals

The experimental animals, i.e., pregnant female Sprague-Dawley rats and C57BL/6 mice (8 to 12 weeks old, with a body weight ranging from 20 g to 30 g) used in the following experiments were purchased from BioLasco Taiwan Co., Ltd. All the experimental animals were housed in an animal room with an independent air conditioning system under an alternating 12-hour light and 12-hour dark cycle, and were provided with water and fed ad libitum. All experimental procedures involving the experimental animals were approved by the Institutional Animal Care and Use Committee (IACUC) of National Yang Ming Chiao Tung University (NYCU), and were carried out according to the Guide for the Care and Use of Laboratory Animals of NYCU.

### 4. Source and Cultivation of Primary Hippocampal Neurons

The primary hippocampal neurons used in the following experiments were isolated from the hippocampus of pups (1 to 3 days old) born from the pregnant female Sprague-Dawley rats according to the method described in Su C. L. et al. (2022), Commun. Biol., 5:1161, and then were incubated in a Petri dish containing a basal medium, i.e., Neurobasal™-A Medium (Gibco, Catalogue no.: 10888-022) supplemented with B-27™ Supplement (Gibco, Catalogue no.: 17504-044) and GlutaMAX™ Supplement (Gibco, Catalogue no.: 35050-061), followed by cultivation in an incubator with culture conditions set at 37° C. and 5% CO₂. On the third day of in vitro cultivation (i.e., after isolation from the hippocampus), 4 μm of 5-fluoro-2′-deoxyuridine (Sigma-Aldrich) was added to the primary hippocampal neurons to inhibit growth of glial cells. The following experiments were conducted within the 5^{th} day to the 14^{th} day of in vitro cultivation.

### General Experimental Procedures

### 1. Magnetic Field Treatment

In the following experiments, the primary hippocampal neurons and experimental animals described in the General Experimental Materials were subjected to magnetic field treatment conducted with reference to Su C. L. et al. (2022), Commun. Biol., 5: 1161 under the following conditions: for the primary hippocampal neurons, an external magnetic field (i.e., an alternating magnetic field) having an amplitude of 50 mT and a frequency of 10 Hz was applied once for 50 seconds using a copper wire coil (number of turns of 2000, wire gauge of 18 AWG, resistance of 7Ω, and inductance of 60 mH) at an alternating current (AC) of 3 A, followed by 50 seconds of rest; while for the experimental animals, an external magnetic field having an amplitude of 50 mT and a frequency of 10 Hz was applied ten times (each application of the external magnetic field was conducted for 30 seconds followed by 30 seconds of rest between two adjacent applications) using four copper wire coil (number of turns of 500, wire gauge of 12 AWG, resistance ranging from 1.02Ω to 1.55Ω, and inductance ranging from 22 mH to 31.6 mH) at an alternating current (AC) of 10 A.

### 2. Measurement of Calcium Ion Influx

In the following experiments, the measurement of calcium ion influx was conducted according to the procedures described in Su C. L. et al. (2022), Commun. Biol., 5: 1161, and the fluo-4 imaging was performed using a fluorescence microscope (Scientifica, Model no.: SS-1000-00). The thus obtained calcium ion fluorescence signal was converted to fluorescence intensity (%) (i.e., ΔF/F₀) according to the procedures described in Gregurec D. et al. (2020), ACS Nano., 14: 8036-8045.

### 3. Statistical Analysis

All the experiments described below were performed at least 3 times, unless otherwise noted. Statistical analysis was conducted using Jeffreys's Amazing Statistics Program (JASP) software (Developer: JASP team). The experimental data of all the test groups are expressed as mean±standard deviation (SD), and were analyzed using paired t-test, so as to assess the differences between the groups. Statistical significance is indicated by p<0.05.

### Example 1. Preparation of BTO and MND, and Evaluation of the Same

### A. Preparation of Neutravidin-Conjugated BTO

First, 20 mg of methoxy polyethylene glycol (mPEG)-silane (average molecular weight of 30000 g/mol, Manufacturer: Creative PEGWorks; Catalogue no.: PSB-2014) was dissolved in 9 mL of 95% ethanol, followed by adding 10 mg of BTO having a particle size of 100 nm as described in Item 1 of the General Experimental Materials. Thereafter, sonication was conducted for 2 hours, and the resultant mixture was subjected to centrifugation at 8500 rpm for 10 minutes to form supernatant and pellet fractions. After that, the supernatant was removed, and the pellet was washed three times with ddH₂O, thereby obtaining mPEG-functionalized BTO.

In addition, 200 μL of 2 mg/ml neutravidin (Thermo Scientific) was mixed with 200 μL of Alexa Fluor 488 dye (Thermo Scientific) for 2 hours. Then, 10 mg of mPEG-functionalized BTO was added, followed by a reaction at 4° C. for 2 hours. The resultant mixture was subjected to centrifugation at a speed ranging from 8300 rpm to 8500 rpm for 3 minutes to form supernatant and pellet fractions. After that, the supernatant was removed, and the pellet was washed three times with ddH₂O, thereby obtaining n-BTO with Alexa Fluor 488 dye.

### B. Preparation of Biotin-Conjugated MND

First, poly(maleic anhydride-alt-1-octadecene) (average molecular weight ranging from 30000 g/mol to 50000 g/mol, Manufacturer: Sigma-Aldrich; Catalogue no.: 419117) was dissolved in 1 mL of chloroform, followed by adding 1 mg of MND having a diameter of 250 nm as described in Item 2 of the General Experimental Materials. Next, sonication was conducted for 1 hour, and the resultant first mixture was subjected to vacuum-drying overnight, followed by addition of 25% TAE (Biomate). Thereafter, sonication was conducted at 80° C. for 3 hours, and the resultant second mixture was subjected to centrifugation at 8500 rpm for 10 minutes to form supernatant and pellet fractions. After that, the supernatant was removed, and the pellet was washed three times with ddH₂O, thereby obtaining PMAO-functionalized MND.

In addition, 100 μL of 30 μM biotin (Sigma-Aldrich, Catalogue no.: MXBIOS 100) was mixed with 10 μL of Alexa Fluor 594 dye (Thermo Scientific) for 2 hours. Then, 10 mg of PMAO-functionalized MND was added, followed by a reaction at 4° C. for 2 hours. The resultant mixture was subjected to centrifugation at a speed ranging from 8300 rpm to 8500 rpm for 3 minutes to form supernatant and pellet fractions. After that, the supernatant was removed, and the pellet was washed three times with ddH₂O, thereby obtaining b-MND with Alexa Fluor 594 dye.

C. Treatment of Primary Hippocampal Neurons With n-BTO and b-MND

First, the primary hippocampal neurons as described in Item 4 of the General Experimental Materials were seeded at a concentration of 7.5×10⁴ cells per well into respective wells of 24-well plates each containing 1 ml of a basal medium, i.e., Neurobasal™-A Medium, and then cultivated in an incubator with culture conditions set at 37° C. and 5% CO₂ for at least 5 days. Next, the primary hippocampal neurons in each well were treated with 70 μg of n-BTO with Alexa Fluor 488 dye and 70 μg of b-MND with Alexa Fluor 594 dye, and then cultivated in the incubator at 37° C. and 5% CO₂ for 15 minutes.

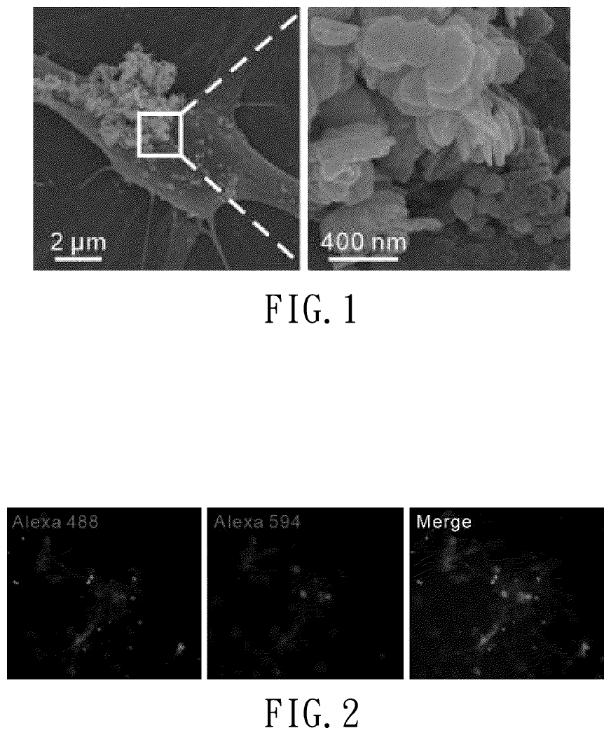

The thus treated primary hippocampal neurons were washed three times with phosphate-buffered saline (PBS), and then subjected to fixation using an appropriate amount of a solution containing 3% glutaraldehyde, 2% paraformaldehyde, and 0.1 M cacodylate, followed by observation and photography using a field emission scanning electron microscope (FE-SEM) (Hitachi, Model no.: SU8220) at a magnification ranging from 2500× to 5000×. The results are shown in FIG. 1.

As shown in FIG. 1, n-BTO is attached to the cell membrane of the primary hippocampal neurons, and such attachment is believed to be promoted by the presence of mPEG, which is a negatively charged molecule, while b-MND is attached to the n-BTO.

In addition, the treated primary hippocampal neurons were subjected to observation and photography using a fluorescence microscope (Scientifica, Model no.: SS-1000-00) at an excitation wavelength of 470 nm and a magnification of 40×, followed by analysis of energy transfer of red fluorescence with respect to the green fluorescence using HCImage (Hamamatsu), so as to determine fluorescence resonance energy transfer (FRET) ratio. The results are shown in FIG. 2.

As shown in FIG. 2, the green fluorescence excited by the n-BTO with Alexa Fluor 488 dye and the red fluorescence excited by the b-MND with Alexa Fluor 594 dye can be observed on the primary hippocampal neurons. In addition, thus obtained FRET ratio is approximately 1.5, indicating that b-MND is indeed attached to the n-BTO, and a distance therebetween (i.e., Förster radius) is less than 3 nm to 6 nm.

### Example 2. Evaluation of BTO in Combination With MND on Magnetic Field-Induced Electrical Stimulation In Vitro

In this example, the efficacy of magnetic field-induced electrical stimulation (also known as magnetoelectric stimulation) of BTO and MND of the present disclosure was evaluated by analyzing the calcium ion influx of cells (with respect to activation of calcium channels on cell membrane) using Fluo-4 Ca²⁺ Imaging Kit (Invitrogen) according to the manufacturer's instructions. In order to avoid the results of this evaluation being affected by the transient receptor potential cation channel (TRPC) that is mechanosensitive, TRPC antagonist, i.e., SKF-96365 was used in the following experiments.

A. Preparation of n-BTO

The procedures and conditions for preparing the n-BTO of this example were substantially similar to those as described in section A of Example 1, except that, in this example, 10 mg of mPEG-functionalized BTO was only reacted with 400 μL of 2 mg/ml neutravidin, and Alexa Fluor 488 dye was omitted.

B. Preparation of b-MND

The procedures and conditions for preparing the b-MND of this example were substantially similar to those as described in section B of Example 1, except that, in this example, 10 mg PMAO-functionalized MND was only reacted with 100 μL of 30 μM biotin, and Alexa Fluor 594 dye was omitted.

### C. Evaluation of Magnetic Field-Induced Electrical Stimulation

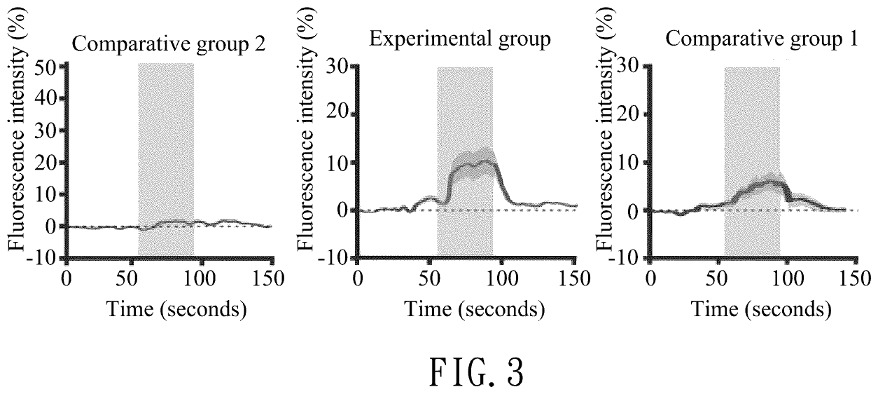

First, the primary hippocampal neurons as described in Item 4 of the General Experimental Materials were seeded at a concentration of 7.5×10⁴ cells per well into respective wells of 24-well plates each containing 500 μL of 1 mM Fluo-4 solution (Invitrogen), and then cultivated in an incubator with culture conditions set at 37° C. and 5% CO₂ for a time period ranging from 15 minutes to 30 minutes. Next, the resultant cultured cells were washed three times using Tyrode's solution containing 50 mMSKF-96365, 125 mM NaCl, 2 nM KCl, 2 mM MgCl₂, 2 mM CaCl₂, 25 mM HEPES and 51 mM D-glucose, and then divided into 3 groups, namely, comparative group 1, comparative group 2, and an experimental group. Next, the cells of the experimental group were treated with 70 μg of n-BTO (dissolved in 500 μL of Tyrode's solution) per well, the cells of the comparative group 1 were treated with 70 μg of b-MND (dissolved in 500 μL of Tyrode's solution) per well, and the cells of the comparative group 2 were treated with Tyrode's solution in a volume equal to that of the cells. Then, the cells in each group were cultivated in the incubator at 37° C. and 5% CO₂ for 5 minutes, followed by washing three times using Tyrode's solution. Thereafter, the cells of a respective one of the experimental group and the comparative group 2 were treated with 70 μg of b-MND (dissolved in 500 μL of Tyrode's solution) per well, while the cells in the comparative group 1 were treated with 70 μg of n-BTO (dissolved in 500 μL of Tyrode's solution) per well. After cultivation in the incubator at 37° C. and 5% CO₂ for 5 minutes, the cells in each group were subjected to magnetic field treatment and measurement of calcium ion influx as respectively described in Items 1 and 2 of the General Experimental Procedures. The results are shown in FIG. 3.

As shown in FIG. 3, during the 50^{th} second to the 100^{th} second of application of the magnetic field, there was no significant change in the fluorescence intensity of the comparative group 2, indicating that SKF-96365 successfully inhibited calcium ion influx caused by the TRPC that is mechanosensitive, such that the primary hippocampal neurons in each group would not be directly stimulated so as to be activated by the torque (that is, conversion of magnetic energy into mechanical energy) generated by the MND under application of the magnetic field. In comparison with the comparative group 2, the fluorescence intensity of the experimental group increased significantly under application of the magnetic field, and such increase was significantly higher than that of the comparative group 1, indicating that the MND, under application of the magnetic field, will generate torque, i.e., conversion of magnetic energy into mechanical energy, and the mechanical energy will be applied to the BTO which is in contact with the primary hippocampal neurons, such that the BTO converts the mechanical energy into an electrical energy to electrically stimulate the primary hippocampal neurons so as to activate the same. In contrast, if the positions of the MND and the BTO relative to the primary hippocampal neurons are reversed (i.e., the MND is positioned between the BTO and the primary hippocampal neurons), the efficiency of electrical stimulation will be greatly reduced.

### Example 3. Evaluation of BTO in Combination With MND on Magnetic Field-Induced Electrical Stimulation In Vivo

In order to determine whether the combination of BTO and MND of the present disclosure, under application of a magnetic field, is capable of electrically stimulate the cells in vivo so as to activate the same, the following experiments were conducted, and the expression of c-Fos, which is a marker of neural activity, was used as an indicator of evaluation.

### Experimental Methods:

First, male C57BL/6 mice (n=3) as described in Item 3 of the General Experimental Materials were anesthetized and then subjected to stereotaxic craniotomy, and then n-BTO and b-MND as obtained in Example 2 were sequentially injected into the amygdala (AP=−0.8 mm; ML=3.05 mm; DV=4.4 mm) on one side of the brain of each mouse using a microinjection syringe (Hamilton; Model no.: 7803-05). To be specific, the n-BTO and the b-MND were first dissolved in PBS, and were administered to each mouse at dosages of 20 μg and 2 μg, respectively, or at dosages of 10 μg and 2 μg, respectively. On the first day after administration, each mouse was subjected to magnetic field treatment as described in Item 1 of the General Experimental Procedures for 90 minutes. Thereafter, the mice were sacrificed by cardiac perfusion, and the brain tissues thereof were removed to analyze the expression of c-Fos as described hereinafter.

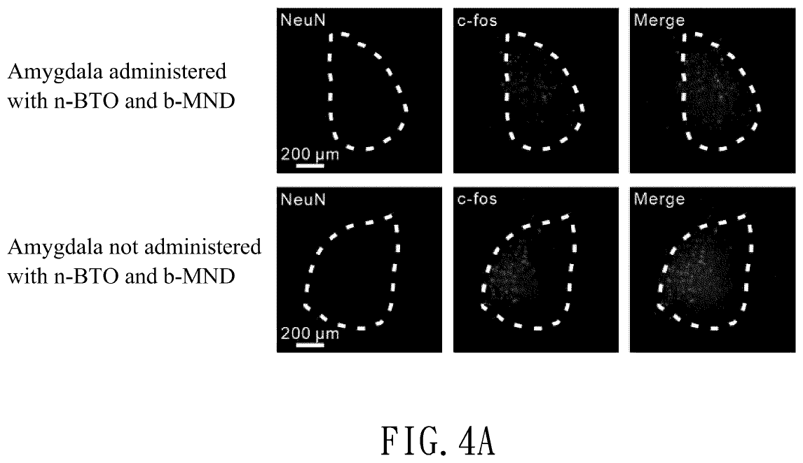

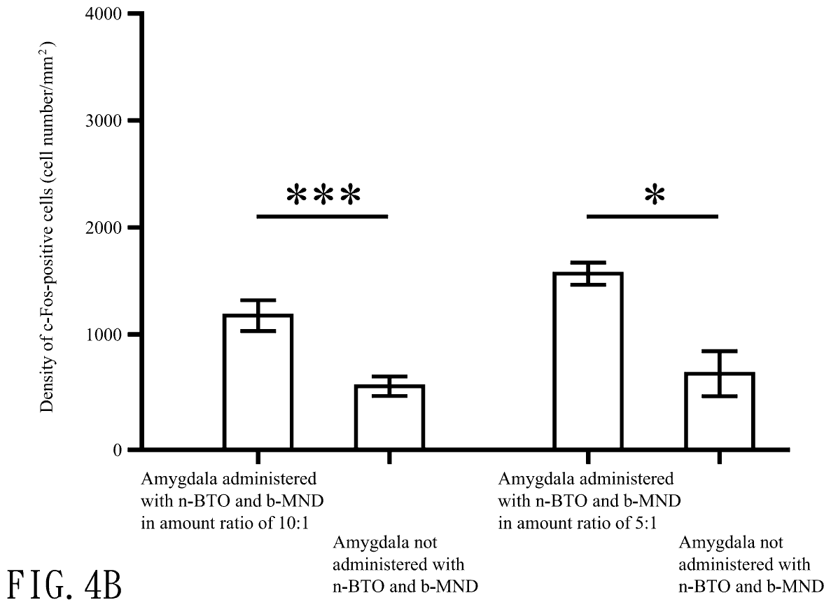

The brain tissues of each mouse were fixed using 4% paraformaldehyde at room temperature for 24 hours, and then embedded in paraffin, followed by sectioning, so as to obtain a tissue section having a thickness of 60 μm. Thereafter, the tissue section was subjected to immunofluorescence staining conducted in accordance to techniques well-known and customary to those skilled in the art using rabbit anti-c-Fos monoclonal antibody (Cell Signaling Technology, Catalogue no.: 9F6#2250) and goat anti-rabbit IgG antibody (Abcam, Catalogue no.: ab150080), which served as the primary antibody and the secondary antibody, respectively, followed by calculation of the density of c-Fos-positive cells (i.e., c-Fos-expressing cells) in the amygdalae on both sides of the brain of each mouse (i.e., including the side of the brain which was administered with n-BTO and b-MND in an amount ratio of 10:1 or 5:1 and another side of the brain which was not administered with n-BTO and b-MND) using ImageJ software. The results are shown in FIGS. 4A and 4B.

### Results:

FIG. 4A shows immunofluorescence staining images of an amygdala on one side of the mouse brain which was administered with n-BTO and b-MND, and those of another amygdala on another side of the mouse brain which was not administered with n-BTO and b-MND, while FIG. 4B shows a graph of the density of c-Fos-positive cells in the amygdala on one side of the mouse brain which was administered with n-BTO and b-MND in an amount ratio of 10:1 or 5:1 and that in the another amygdala on the another side of the mouse brain which was not administered with n-BTO and b-MND. As shown in FIG. 4B, the density of c-Fos-positive cells (i.e., c-Fos-expressing cells) in the amygdala on one side of the mouse brain which was administered with n-BTO and b-MND in an amount ratio of 10:1 or 5:1 was significantly higher than that in the another amygdala on the another side of the mouse brain which was not administered with n-BTO and b-MND. These result shows that, the BTO in combination with the MND of the present disclosure are capable of electrically stimulate the neurons in the amygdala of the mouse brain so as to improve neuronal activity.

### Example 4. Evaluation of the Effect of Different Sizes of MND and BTO on Magnetic Field-Induced Electrical Stimulation

### A. Evaluation of the Effect of Different Particle Sizes of BTO on Magnetic Field-Induced Electrical Stimulation

### Experimental Methods:

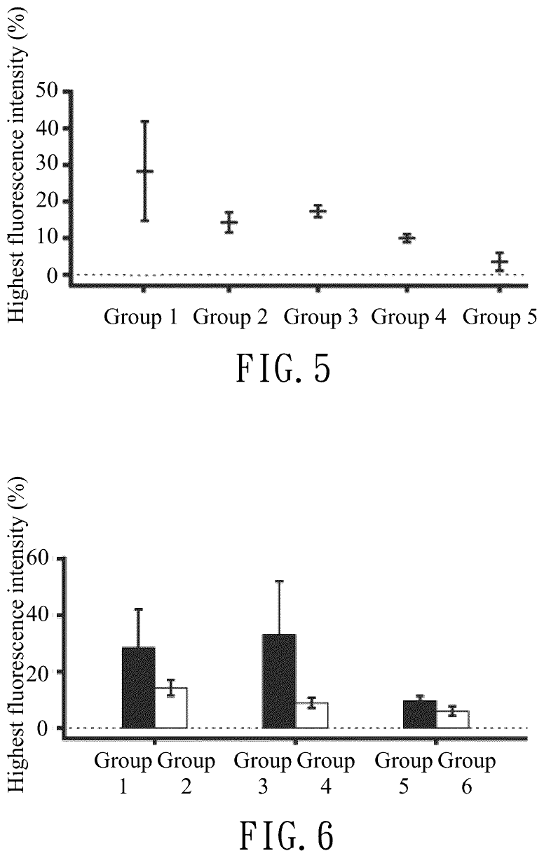

The procedures and conditions for preparing the n-BTO of this example were substantially similar to those as described in section A of Example 2 above, except that, in this example, n-BTO having different particle sizes, i.e., 50 nm, 100 nm, 200 nm, 300 nm and 500 nm were used as starting material, so as to obtain n-BTO having different particle sizes, i.e., n-BTO₅₀, n-BTO₁₀₀, n-BTO₂₀₀, n-BTO₃₀₀ and n-BTO₅₀₀ hereinafter. In addition, the b-MND having a diameter of 250 nm of this example, i.e., b-MND₂₅₀ hereinafter, were prepared using procedures and conditions similar to those as described in section B of Example 2 above. Thereafter, in combination with b-MND₂₅₀, a respective one of the n-BTO₅₀, n-BTO₁₀₀, n-BTO₂₀₀, n-BTO₃₀₀ and n-BTO₅₀₀ in each group as shown in Table 1 below were subjected to evaluation of magnetic field-induced electrical stimulation according to the procedures and conditions applied to the experimental group as described in section C of Example 2 above, followed by determination of the highest fluorescence intensity, i.e., the highest ΔF/F₀. The results are shown in FIG. 5.

TABLE 1

| Group | BTO | MND |
|---|---|---|
| 1 | n-BTO₅₀ | b-MND₂₅₀ |
| 2 | n-BTO₁₀₀ | |
| 3 | n-BTO₂₀₀ | |
| 4 | n-BTO₃₀₀ | |
| 5 | n-BTO₅₀₀ | |

### Results:

FIG. 5 is a graph showing the highest fluorescence intensity determined in each group after magnetic field-induced electrical stimulation in cells treated with n-BTO having different particle sizes in combination with b-MND₂₅₀. As shown in FIG. 5, when used in combination with b-MND₂₅₀, the n-BTO having different particle sizes exhibited varying degrees of efficacy in magnetic field-induced electrical stimulation, in which a respective one of n-BTO₅₀, n-BTO₁₀₀ and n-BTO₂₀₀ in combination with b-MND₂₅₀ (i.e., groups 1 to 3) exhibited an excellent efficacy, whereas n-BTO₅₀₀ in combination with b-MND₂₅₀ (i.e., group 5) exhibited a relatively poor efficacy. Therefore, n-BTO₅₀ and n-BTO₁₀₀ were selected for the following experiments.

### B. Evaluation of the Effect of Different Diameters of MND on Magnetic Field-Induced Electrical Stimulation

### Experimental Methods:

The procedures and conditions for preparing the b-MND of this example were substantially similar to those as described in section B of Example 2 above, except that, in this example, b-MND having different diameters, i.e., 150 nm, 200 nm, and 250 nm were used as starting material, so as to obtain b-MND having different diameters, i.e., b-MND₁₅₀, b-MND₂₀₀, and b-MND₂₅₀ hereinafter. Thereafter, in combination with n-BTO₅₀ or n-BTO₁₀₀, a respective one of the b-MND₁₅₀, b-MND₂₀₀, and b-MND₂₅₀ in each group as shown in Table 2 below were subjected to evaluation of magnetic field-induced electrical stimulation according to the procedures and conditions applied to the experimental group as described in section C of Example 2 above, followed by determination of the highest fluorescence intensity, i.e., the highest ΔF/F₀. The results are shown in FIG. 6.

TABLE 2

| Group | BTO | MND |
|---|---|---|
| 1 | n-BTO₅₀ | b-MND₂₅₀ |
| 2 | n-BTO₁₀₀ | |
| 3 | n-BTO₅₀ | b-MND₂₀₀ |
| 4 | n-BTO₁₀₀ | |
| 5 | n-BTO₅₀ | b-MND₁₅₀ |
| 6 | n-BTO₁₀₀ | |

### Results:

FIG. 6 is a graph showing the highest fluorescence intensity determined in each group after magnetic field-induced electrical stimulation in cells treated with n-BTO having different particle sizes in combination with b-MND having different diameters. As shown in FIG. 6, various combinations of the n-BTO and the b-MND exhibited varying degrees of efficacy in magnetic field-induced electrical stimulation, in which n-BTO₅₀ in combination with either b-MND₂₅₀ or b-MND₂₀₀ (i.e., groups 1 and 3), and n-BTO₁₀₀ in combination with b-MND₂₅₀ (i.e., group 2) exhibited an excellent efficacy, whereas n-BTO₁₀₀ in combination with b-MND₁₅₀ (i.e., group 6) exhibited a relatively poor efficacy.

These results showed that cells can be subjected to magnetic field-induced electrical stimulation using BTO having different particle sizes (in a specific range) in combination with MND having different diameters (in a specific range), particularly when a ratio of the particle size of the BTO to the diameter of the MND ranges from 1:2.5 to 1:5.

In summary, since the combination of the piezoelectric nanoparticle and the magnetic nanodisc of the present disclosure is capable of electrically stimulate the cells under application of a magnetic field, the applicant believes that the combination of the piezoelectric nanoparticle and the magnetic nanodisc has a great potential to be developed as a product for magnetic field-induced electrical stimulation of cells.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is(are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A nanomaterial composition for magnetic field-induced electrical stimulation of cells, the nanomaterial composition comprising:
a piezoelectric nanoparticle which is conjugated to a first molecule of a specific binding molecule pair and which is coated with a cell-binding molecule; and
a magnetic nanodisc which is conjugated to a second molecule of the specific binding molecule pair, the magnetic nanodisc being attached to the piezoelectric nanoparticle through bonding of the second molecule and the first molecule,
wherein the magnetic nanodisc converts a magnetic energy into a mechanical energy in the presence of an external magnetic field, and the mechanical energy is then applied to the piezoelectric nanoparticle that is in contact with cells via the cell-binding molecule, such that the piezoelectric nanoparticle converts the mechanical energy into an electrical energy, so as to electrically stimulate the cells.

2. The nanomaterial composition as claimed in claim 1, wherein the piezoelectric nanoparticle is selected from the group consisting of BaTiO₃ nanoparticle, MoS₂ nanoparticle, KNbO₃ nanoparticle, LiNbO₃ nanoparticle, BiFeO₃ nanoparticle, Pb(Zr, Ti)O₃ nanoparticle, and combinations thereof.

3. The nanomaterial composition as claimed in claim 1, wherein the magnetic nanodisc is selected from the group consisting of Fe₃O₄ nanodisc, γ-Fe₂O₃ nanodisc, CoFe₂O₄ nanodisc, Co_{1.2}Fe_{1.8}O₄ nanodisc, Ni(OH)₂ nanodisc, and combinations thereof.

4. The nanomaterial composition as claimed in claim 1, wherein the piezoelectric nanoparticle has a particle size ranging from 50 nm to 500 nm.

5. The nanomaterial composition as claimed in claim 1, wherein the magnetic nanodisc has a diameter ranging from 150 nm to 250 nm.

6. The nanomaterial composition as claimed in claim 1, wherein a ratio of a particle size of the piezoelectric nanoparticle to a diameter of the magnetic nanodisc ranges from 1:2.5 to 1:5.

7. The nanomaterial composition as claimed in claim 1, wherein the specific binding molecule pair is selected from the group consisting of neutravidin-biotin pair, avidin-biotin pair, antigen-antibody pair, ligand-receptor pair, DNA-DNA pair, DNA-binding protein-DNA pair, click chemistry reactants, and combinations thereof.

8. The nanomaterial composition as claimed in claim 1, wherein the first molecule of the specific binding molecule pair is neutravidin, and the second molecule of the specific binding molecule pair is biotin.

9. The nanomaterial composition as claimed in claim 1, wherein the cells are selected from the group consisting of neuronal cells, cancer cells, osteocytes, vascular endothelial cells, muscle cells, peritoneal mesothelial cells, and combinations thereof.

10. The nanomaterial composition as claimed in claim 9, wherein the cells are neuronal cells.

11. The nanomaterial composition as claimed in claim 1, wherein the cell-binding molecule is selected from the group consisting of a cell-specific antibody, a cell-affinitive molecule, and a combination thereof.

12. The nanomaterial composition as claimed in claim 11, wherein the cell-specific antibody is selected from the group consisting of a neuronal cell-specific antibody, a cancer cell-specific antibody, and a combination thereof.

13. The nanomaterial composition as claimed in claim 11, wherein the cell-affinitive molecule is selected from the group consisting of methoxy polyethylene glycol (mPEG)-silane, polyethylene glycol (PEG)-NH₂, PEG-COOH, poly (maleic anhydride-alt-1-octadecene (PMAO), PMAO-PEG, polyvinylpyrrolidone (PVP), and combinations thereof.

14. A method for magnetic field-induced electrical stimulation of cells in a subject, comprising:
administering to a subject, a nanomaterial composition as claimed in claim 1, such that a piezoelectric nanoparticle, which is conjugated to a first molecule of a specific binding molecule pair and which is coated with a cell-binding molecule, is in contact with cells in the subject via the cell-binding molecule, and such that a magnetic nanodisc, which is conjugated to a second molecule of the specific binding molecule pair, is attached to the piezoelectric nanoparticle through bonding of the second molecule and the first molecule; and
applying an external magnetic field to the subject, such that the magnetic nanodisc converts a magnetic energy into a mechanical energy, and the mechanical energy is then applied to the piezoelectric nanoparticle that is in contact with the cells, such that the piezoelectric nanoparticle converts the mechanical energy into an electrical energy, so as to electrically stimulate the cells.

15. The method as claimed in claim 14, wherein the piezoelectric nanoparticle is selected from the group consisting of BaTiO₃ nanoparticle, MoS₂ nanoparticle, KNbO₃ nanoparticle, LiNbO₃ nanoparticle, BiFeO₃ nanoparticle, Pb(Zr, Ti)O₃ nanoparticle, and combinations thereof.

16. The method as claimed in claim 14, wherein the magnetic nanodisc is selected from the group consisting of Fe₃O₄ nanodisc, γ-Fe₂O₃ nanodisc, CoFe₂O₄ nanodisc, Co_{1.2}Fe_{1.8}O₄ nanodisc, Ni(OH)₂ nanodisc, and combinations thereof.

17. The method as claimed in claim 14, wherein the piezoelectric nanoparticle has a particle size ranging from 50 nm to 500 nm.

18. The method as claimed in claim 14, wherein the magnetic nanodisc has a diameter ranging from 150 nm to 250 nm.

19. The method as claimed in claim 14, wherein a ratio of a particle size of the piezoelectric nanoparticle to a diameter of the magnetic nanodisc ranges from 1:2.5 to 1:5.

20. The method as claimed in claim 14, wherein the specific binding molecule pair is selected from the group consisting of neutravidin-biotin pair, avidin-biotin pair, antigen-antibody pair, ligand-receptor pair, DNA-DNA pair, DNA-binding protein-DNA pair, click chemistry reactants, and combinations thereof.

21. The method as claimed in claim 14, wherein the first molecule of the specific binding molecule pair is neutravidin, and the second molecule of the specific binding molecule pair is biotin.

22. The method as claimed in claim 14, wherein the cells are selected from the group consisting of neuronal cells, cancer cells, osteocytes, vascular endothelial cells, muscle cells, peritoneal mesothelial cells, and combinations thereof.

23. The method as claimed in claim 22, wherein the cells are neuronal cells.

24. The method as claimed in claim 14, wherein in order to enhance affinity to the cells, the piezoelectric nanoparticle is further conjugated to a substance selected from the group consisting of a cell-specific antibody, a cell-affinitive molecule, and a combination thereof.

25. The method as claimed in claim 14, wherein the external magnetic field has an amplitude of not greater than 200 mT.

26. The method as claimed in claim 14, wherein the external magnetic field has a frequency of not greater than 140 Hz.

27. The method as claimed in claim 14, wherein the piezoelectric nanoparticle and the magnetic nanodisc of the nanomaterial composition are simultaneously administered to the subject.

28. The method as claimed in claim 14, wherein the piezoelectric nanoparticle and the magnetic nanodisc of the nanomaterial composition are sequentially administered to the subject.
